(19) United States
(12) Patent Application Publication
(10) Pub. No.: US 2018/0120277 A1
(43) Pub. Date: May 3, 2018

(54) APPARATUS AND METHOD FOR GENERATION OF OLFACTORY INFORMATION CAPABLE OF CALIBRATION BASED ON PATTERN RECOGNITION MODEL

(71) Applicant: ELECTRONICS AND TELECOMMUNICATIONS RESEARCH INSTITUTE, Daejeon (KR)

(72) Inventors: Sung June CHANG, Daejeon (KR); Jun Seok PARK, Daejeon (KR); Hae Ryong LEE, Daejeon (KR); Jong Woo CHOI, Daejeon (KR)

(21) Appl. No.: 15/426,328

(22) Filed: Feb. 7, 2017

(30) Foreign Application Priority Data
Oct. 31, 2016 (KR) 10-2016-0143696

(51) Int. Cl. G01N 33/00

(52) U.S. Cl. CPC G01N 33/0034; G06F 3/147; G01N 33/007

(57) ABSTRACT

An apparatus and method for generating olfactory information, which relate to a method of expressing the capability of an electronic nose and delivery of a recognized odor in a virtual reality system. The apparatus includes a plurality of gas sensors configured to recognize real-world odors and acquire raw data of a result of recognizing the real-world odors and a processor configured to generate a calibration model determination result of each of the plurality of gas sensors by applying a calibration model allocated to each of the plurality of gas sensors to measurement data of each of the plurality of gas sensors, compare the calibration model determination results of the plurality of gas sensors with each other, and determine whether to calibrate an olfactory recognition model for recognizing the real-world odors, based on a result of the comparison between the calibration model determination results.

### CROSS-REFERENCE TO RELATED APPLICATION

This application claims priority to Korean Patent Application No. 2016-0143696, filed Oct. 31, 2016 in the Korean Intellectual Property Office (KIPO), the entire content of which is hereby incorporated by reference.

### BACKGROUND

### 1. Technical Field

Example embodiments of the present invention relate in general to a method of expressing the capability of an electronic nose and delivery of a recognized odor in a virtual reality system based on MPEG-V (Media Context and Control) and more specifically to MPEG-V technology in which a virtual reality system provides interoperability between a virtual world and a real world.

### 2. Description of Related Art

The present invention relates to a method of expressing the capability of an electronic nose and delivery of a recognized odor in a virtual reality system based on MPEG-V (Media Context and Control), and more particularly, to MPEG-V technology in which a virtual reality system provides interoperability between a virtual world and a real world.

An electronic nose (e-nose) is used as a sensor intended to detect gas or particles that cause odors in a real world. In the real world, odors are detected based on the concentration of gas or the concentration of particles that cause odors in a physical, chemical, or biological manner.

Efforts are underway through MPEG-V standardization meetings to represent and express olfactory information detected by an e-nose sensor in a virtual world or another real world.

Likewise, there is a need to develop a type of data for sharing olfactory information between a virtual world and a real world, which is advanced and standardized through MPEG-V standardization meetings.

### SUMMARY

Accordingly, example embodiments of the present invention are provided to substantially obviate one or more problems due to limitations and disadvantages of the related art.

The present invention is directed to providing interoperability between a virtual world and a real world by recognizing real-world odors within the scope of MPEG-V and delivering the real-world odors to the virtual world.

The present invention is directed to generating and delivering detailed information while delivering real-world odors to a virtual world. Generally, semiconductor-type gas sensors used for an e-nose have low prices and stable performance, but have a disadvantage in terms of selectivity that indicates a selective response only to a specified gas. Accordingly, the amount of information is generally increased by using several sensor modules at the same time. In this case, although gas sensors are degraded, the degradation of gas sensors cannot be easily found. That is, it is difficult to accurately recognize a true determination result using only a value measured by a semiconductor-type gas sensor.

The present invention is also directed to generating a gas determination pattern recognition model for receiving an input of a value measured by a gas sensor and estimating a true determination result to discover an accurate gas determination result.

Semiconductor-type gas sensors may be degraded due to aging over time, and thus an undesired change in sensor values may occur. In the related art, it is impossible to know when the degradation will occur due to aging. Accordingly, there is an inconvenience that gas sensors should be periodically collected, checked, and calibrated in an offline state because the gas sensors may be degraded due to aging at any time.

According to an embodiment of the present invention, processors included in gas sensors may identify a gas sensor that needs to be calibrated by using a pattern recognition model, determine when the gas sensor will be calibrated, and calibrate the gas sensor using a correlation between a plurality of gas sensors, without collecting the gas sensor in an offline state. That is, the present invention is also directed to providing an olfactory information generation apparatus configured to determine necessity and time of calibration through self-learning of a gas sensor and calibrate measured values.

In order to achieve the above objectives, an olfactory information generation method according to an embodiment of the present invention assumes that human perception intensity with respect to concentration of chemical material detected by an electronic nose is recorded. In this case, information determined as a quantitative value felt by human olfaction is generated as information having an XML format or the like.

In some example embodiments, an olfactory information generation method for generating olfactory information suitable for sharing between a real world and at least one virtual world includes generating a calibration model determination result of each of a plurality of gas sensors included in a sensor device (referring to any sensor device including gas sensor modules) configured to recognize real-world odors by applying a calibration model (a data calibration pattern recognition model) allocated to each of the plurality of gas sensors to measurement data of each of the plurality of gas sensors; comparing the calibration model determination results of the plurality of gas sensors with each other (by quantifying and calculating a degree of similarity between the calibration model determination results); and determining whether to calibrate an olfactory recognition model (a gas determination pattern recognition model) for recognizing the real-world odors, based on a result of the comparison between the calibration model determination results.

In other example embodiments, an olfactory information generation apparatus includes a plurality of gas sensors configured to recognize real-world odors and acquire raw data of a result of recognizing the real-world odors; and a processor configured to generate a calibration model determination result of each of the plurality of gas sensors by applying a calibration model allocated to each of the plurality of gas sensors to measurement data of each of the plurality of gas sensors, compare the calibration model determination results of the plurality of gas sensors with each other, and determine whether to calibrate an olfactory recognition model for recognizing the real-world odors, based on a result of the comparison between the calibration model determination results.

The olfactory information generation apparatus according to an embodiment of the present invention may be implemented by using a plurality of sensor modules as a semiconductor-type gas sensor. In this case, the olfactory information generation apparatus may generate a data calibration pattern recognition model of each of a plurality of sensors or each of a plurality of sensor groups by using measurement data obtained in a divided manner by the plurality of sensors.

The olfactory information generation apparatus according to an embodiment of the present invention may apply real-time data of a gas sensor to a plurality of divided data calibration pattern recognition models online or on-site. In this case, the olfactory information generation apparatus may obtain a determination result for each data calibration pattern recognition model by applying real-time measurement data of the gas sensors to the data calibration pattern recognition model on the basis of the same time.

The olfactory information generation apparatus according to an embodiment of the present invention may determine a sensor to be calibrated from among the sensors and also determine a calibration time by utilizing a determination result obtained by a plurality of divided data calibration pattern recognition models. That is, the olfactory information generation apparatus may digitize a degree of matching between determination results of a plurality of divided data calibration pattern recognition models obtained at the same time and determine that calibration is needed or that calibration is possible when the digitized value exceeds a reference.

In this case, the olfactory information generation apparatus according to an embodiment of the present invention may collect a calibration data group using a determination result obtained by a plurality of divided data calibration pattern recognition models. When determination results of the plurality of divided data calibration pattern recognition models obtained at the same time have a high degree of matching but do not completely match each other, the olfactory information generation apparatus may collect corresponding data to generate a calibration data group. Among a group of sensors classified by the calibration data group, a sensor having a degree of similarity between the determination results lower than a reference is regarded as a sensor to be calibrated.

The olfactory information generation apparatus according to an embodiment of the present invention may generate one gas determination pattern recognition model using the calibration data group and replace a gas determination pattern recognition model determined to need calibration with the generated gas determination pattern recognition model. The olfactory information generation apparatus may generate a new gas determination pattern recognition model using a calibration data group generated online or on-site, calibrate an old gas determination pattern recognition model, and replace the old pattern recognition model with the new pattern recognition model.

### BRIEF DESCRIPTION OF DRAWINGS

Example embodiments of the present invention will become more apparent by describing in detail example embodiments of the present invention with reference to the accompanying drawings, in which:

FIG. 1 is a block diagram showing an olfactory information generation apparatus according to an embodiment of the present invention;

FIGS. 2 and 3 are diagrams showing examples of a detailed configuration of a sensor 110 of FIG. 1;

FIGS. 4 and 5 are flowcharts showing an olfactory information generation method according to an embodiment of the present invention;

FIG. 6 is a diagram showing gas concentration areas corresponding to a plurality of gas sensors according to an embodiment of the present invention;

FIG. 7 is a diagram showing a process in which a plurality of gas sensors generate a first group and a second group according to a degree of similarity between determination results and calibrate a determination result of the second group according to an embodiment of the present invention; and

FIG. 8 is a diagram in which “Semantics of the EnoseSensorType” of semantics of the Enose Sensor Type is suggested according to an embodiment of the present invention.

### DESCRIPTION OF EXAMPLE EMBODIMENTS

These and other objectives and features of the present invention will become more fully apparent from the following description taken in conjunction with the accompanying drawings.

Example embodiments of the present invention will be described in detail with reference to the accompanying drawings. Moreover, detailed descriptions related to well-known functions or configurations will be ruled out in order not to unnecessarily obscure subject matters of the present invention. Sizes of elements in the drawings may be exaggerated for convenience of explanation.

However, the present invention is not restricted or limited to the embodiments. Like reference numerals in the drawings denote like elements.

A general virtual world processing system included as an element of the present invention may correspond to an engine, a virtual world, and a real world. In the real world, an e-nose device for detecting information regarding the real world or a scent display device for implementing information regarding the virtual world are included. Also, in the virtual world, a scent medium playback device for playing content including the virtual world itself, which is implemented by a program, or scent information that may be implemented in the real world may be included.

For example, an e-nose device may detect information regarding real-world odors and the capability and specifications of the e-nose device and transmit this information to an engine. Alternatively, the e-nose device may include an e-nose capability type, which is a part for delivering the capability and specifications of the e-nose device to an engine, odor sensor technology CS, which is a part for describing the type of a sensor needed to define the e-nose capability type, and an e-nose sensed info type, which is a part for delivering information recognized by the e-nose device to the engine.

The engine may transmit sensed information to the virtual world. In this case, the sensed information is applied to the virtual world. Thus, an effect corresponding to the e-nose sensed info type corresponding to a real-world odor may be implemented in the virtual world.

An effect event generated in the virtual world may be driven by the scent display device in the real world. Virtual information (sensory effect), which is information regarding an effect event generated in the virtual world, may be transmitted to the engine. Also, virtual world characteristics (VW object characteristics) may be transmitted between the virtual world and the engine.

Provision of the scent display device present in the real word and a user's preference will be described within the scope of MPEG-V. The scent display device is present in the real world and configured to perform synchronization with content in the virtual world and make the user feel the scent by displaying the scent to the user. To this end, a part for delivering the capability and specifications of the scent display device to the engine is defined as a scent capability type. Also, a part for providing the user's preference in order to correct a difference in characteristics between the scent provided by the scent display device and the scent felt by the user is defined as a scent preference type. Also, a command part for displaying the scent by the scent display device is defined as a scent effect.

A generalized virtual world processing method included as a portion of a configuration of the present invention may be performed by mutually transmitting olfactory information regarding a virtual world, a real world, and another virtual world between the real world and the real world or between the virtual world and the other virtual world and expressing the olfactory information through the scent display device. The generalized virtual world processing method may include acquiring virtual information, which is the olfactory information regarding the virtual world, acquiring real information, which is the olfactory information regarding the real world, through a reality recognition unit that is a device for recognizing an odor, providing the virtual information to the real world or the other virtual world, providing the real information to the virtual world or the other virtual world, and displaying a scent to a user through the scent display device on the basis of the virtual information and the real information. The real information includes an e-nose capability type, which is a part for delivering the capability and specifications of an e-nose device that is the reality recognition unit to an engine, odor sensor technology CS, which is a part for describing the type of a sensor needed to define the e-nose capability type, and an e-nose sensed info type, which is a part for delivering information recognized by the e-nose device to the engine.

Also, steps of defining a scent capability type, which is a part for delivering the capability and specifications of a scent display device configured to display a scent, defining a scent preference type, which is a part for providing a user's preference in order to correct a difference in characteristics between the scent provided by the scent display device and the scent felt by the user, and displaying scent effect, which is a command part for instructing the scent display device to display the scent are included.

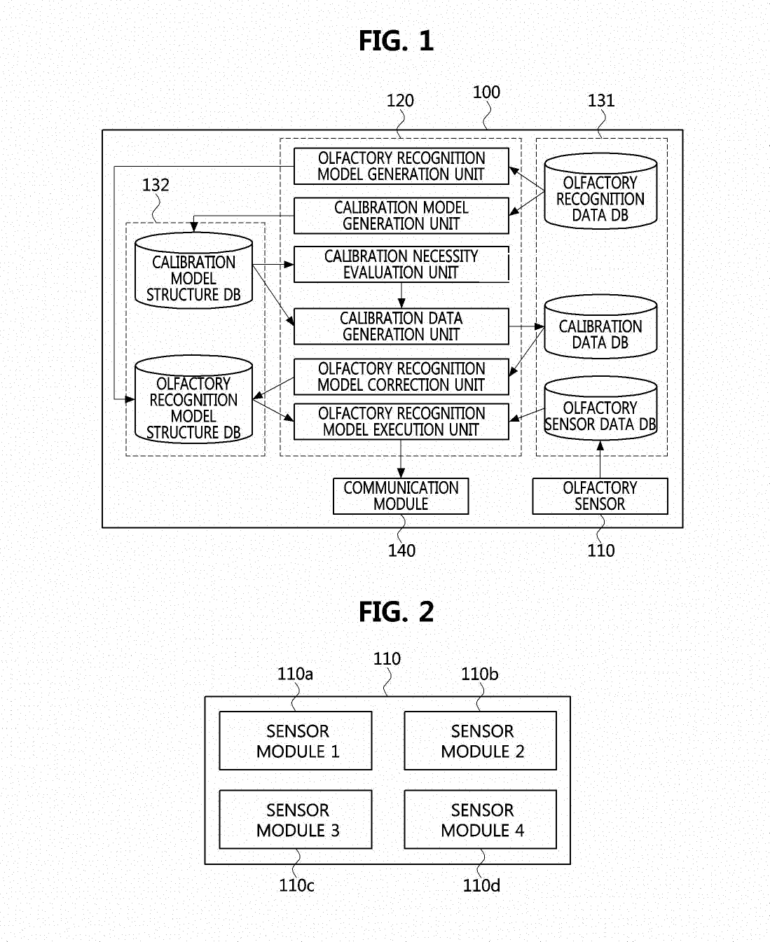

FIG. 1 is a block diagram showing an olfactory information generation apparatus according to an embodiment of the present invention.

An olfactory information generation apparatus 100 of FIG. 1 may be implemented in the form of an e-nose or may be installed in cooperation with a gas sensor. The olfactory information generation apparatus 100 includes a sensor 110, a processor 120, databases 131 and 132, a communication module 140. In this case, the databases 131 and 132 may store any type of data in the olfactory information generation apparatus 100. However, only recent data and model information may be stored in the apparatus 100, and past information may be backed up in an external server and called whenever necessary.

Although not shown in FIG. 1, a user interface such as a button or switch for inputting a power on/off command from the outside may be additionally included. In addition, a user interface such as a keypad, a touchscreen, and a microphone for receiving an input of a simple operation command may be added.

The sensor 110 recognizes a real-world odor. A flow of gas particles constituting the real-world odor may be detected by the sensor 110. In this case, the sensor 110 may be implemented as a combination of a plurality of gas sensors for detecting a specified type of gas. The plurality of gas sensors may detect different types of gas. Also, a plurality of gas sensors corresponding to different concentration ranges of the same type of gas may be included.

The sensor 110 acquires a result obtained by detecting the real-world odor as raw data. The raw data includes quantitative information and qualitative information regarding gas that is actually detected. The quantitative information may be the concentration of gas or the concentration of gas over time, and the qualitative information may include information regarding the type of gas and a situation in which the gas is detected.

The processor 120 may convert the raw data for the real-world odor into expression data including evaluation of a quantitative value of the real-world odor. In this case, the quantitative evaluation may be performed on the basis of information on the sensory evaluation of the real-world odor felt by a human. Information on the sensory evaluation of each type of gas may be stored in the database 131.

The processor 120 generates real-world olfactory information including raw data and expression data. The expression data includes information regarding a threshold of specified gas that humans can feel. In other words, the expression data may include information regarding a gas concentration interval that is imperceptible by humans.

The sensor 110 may track a gas concentration over time and store the gas concentration in the database 131 along with a timestamp. Raw data over time, which is generated by the sensor 110, may be delivered to the processor via the database 131. The processor 120 may convert the raw data over time into quantitative evaluation information according to a gas concentration interval. The processor 120 may generate quantitative evaluation information over time as the expression data.

The communication module 140 may deliver the expression data generated by the processor 120 and the raw data generated by the sensor 110 to an external server or a relay device. The processor 120 may generate XML-formatted olfactory information including the raw data and the expression data. The communication module 140 may deliver the XML-formatted olfactory information instead of delivering the raw data and the expression data.

The communication module 140 may deliver olfactory information generated in real time to the outside. However, the olfactory information may be generated and stored in the database 131 and delivered to the outside by the communication module 140 at certain time intervals.

The raw data detected by the sensor 110 is stored in an olfactory sensor data DB. The raw data may be delivered from the olfactory sensor data DB to an olfactory recognition model execution unit in the processor 120 and used to generate a determination value through an olfactory recognition model.

An olfactory recognition data DB provides data to an olfactory recognition model generation unit and a calibration model generation unit in the processor 120. That is, the olfactory recognition data DB may be involved in a process of generating a calibration model and an olfactory recognition model.

An olfactory recognition model generated by the olfactory recognition model generation unit is stored in an olfactory recognition model structure DB. A calibration model generated by the calibration model generation unit may be stored in a calibration model structure DB.

A calibration necessity evaluation unit in the processor 120 may receive calibration model determination values for sensor modules from the calibration model stored in the calibration model structure DB, calculate correlations between the calibration model determination values, and determine the necessity and possibility of calibration.

A calibration data generation unit may classify the calibration model determination values into a first group with high correlation and a second group with low correlation on the basis of the correlations between the calibration model determination values. Since the calibration model determination values correspond to the respective sensor modules, the first group and the second group may be regarded as being used to classify the sensor modules.

Sensor modules belonging to the first group with high correlation provide reference information through the calibration process, and sensor modules belonging to the second group with low correlation are to be calibrated.

The calibration data generation unit may mix the calibration model determination values of the first group and the second group to generate a calibration data group of the olfactory recognition model to be calibrated. The calibration data group may be stored in the calibration data DB.

An olfactory recognition model correction unit may calibrate the olfactory model using the calibration data group. The olfactory recognition model correction unit may store a new olfactory recognition model structure obtained through calibration and updating in the olfactory recognition model structure DB.

The olfactory recognition model execution unit may receive raw data of olfactory sensor data from the olfactory sensor data DB, apply the calibrated olfactory recognition model structure, and generate determination values obtained through the application as final data of an olfactory sensor. The generated final data may be delivered to the outside via the communication module 140.

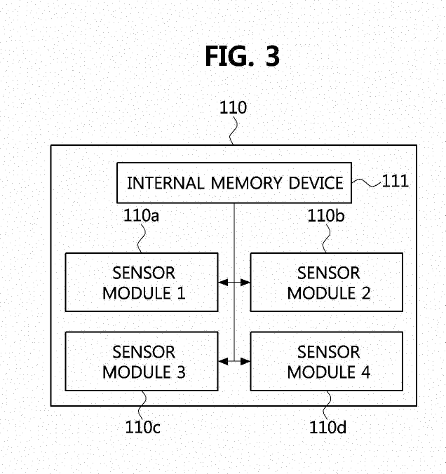

FIGS. 2 and 3 are diagrams showing examples of a detailed configuration of the sensor 110 of FIG. 1. FIG. 2 shows an example in which four sensor modules 110a, 110b, 110c, and 110d are included in the sensor 110, and FIG. 3 shows an example in which an internal memory device 111 is included in the sensor 110 in addition to the four sensor modules 110a, 110b, 110c, and 110d. The internal memory device 111 is a separate module that is distinct from the databases 131 and 132 outside the sensor 110.

Referring to FIGS. 1 to 3 again, the sensor modules 110a, 110b, 110c, and 110d in the sensor 110 recognize real-world odors and acquire raw data of a result of recognizing the real-world odors.

The processor 120 applies a calibration model (a pattern recognition model for data calibration) allocated to each of the sensor modules 110a, 110b, 110c, and 110d in the sensor 110 to measurement data of each of the sensor module 110a, 110b, 110c, and 110d to generate a calibration model determination result of each of the sensor modules 110a, 110b, 110c, and 110d. The processor 120 compares the calibration model determination results of the sensor modules 110a, 110b, 110c, and 110d with each other and determines whether to calibrate an olfactory recognition model (a gas determination pattern recognition model) that recognizes real-world odors. The pattern recognition model may be implemented using a knowledge-based data mining technique, a neural network algorithm, a multiple linear regression technique, etc. Also, a method for improving performance by normalizing and binarizing input variables of a multi-variable pattern recognition model as necessary is already known in Korean Patent No. 10-1638368.

The processor 120 may calculate a correlation (quantification/qualification of a degree of matching between the determination results) indicating a degree of similarity between the calibration model determination results of the sensor modules 110a, 110b, 110c, and 110d. The processor 120 may determine whether the correlation is greater than or equal to a reference in order to determine whether to correct the olfactory recognition model. When it is determined that the correlation between the calibration model determination results of the sensor modules 110a, 110b, 110c, and 110d is greater than or equal to the reference, the processor 120 may determine that the olfactory recognition model can be calibrated.

The processor 120 may select a first group including at least two gas sensor modules having a correlation greater than or equal to the reference and a second group including at least one gas sensor having a correlation less than the reference from among the sensor modules 110a, 110b, 110c, and 110d. When determination results of the divided calibration models obtained at the same time have a high degree of matching but do not completely match each other, a calibration data group may be generated by collecting this data.

The processor 120 may generate the calibration data group for calibrating the olfactory recognition model by using calibration model determination results and measurement data of the gas sensors included in the first group and the second group In this case, the first group may be a group having the correlation between the determination results higher than or equal to the reference, which is a calibration criterion, and the second group may be a group having the correlation between the determination results lower than the reference, which is a calibration target.

The processor 120 may generate the calibration data group including a pattern for calibrating the calibration model determination result of the second group by using a difference between the calibration model determination result of the first group and the calibration model determination result of the second group.

When it is determined to calibrate the olfactory recognition model, the processor 120 may generate one new olfactory recognition model using the calibration data group and replace the existing olfactory recognition model with the new olfactory recognition model. That is, the processor 120 may generate the calibration data group of the olfactory recognition result by using the calibration model determination results and the measurement data of the sensor modules 110a, 110b, 110c, and 110d. The processor 120 may update the olfactory recognition model by calibrating the olfactory recognition model using the calibration data group.

In this case, the calibration model determination results to be compared are obtained by applying the calibration model allocated to each of the sensor modules 110a, 110b, 110c, and 110d to the measurement data of the sensor modules 110a, 110b, 110c, and 110d during the same first reference time interval and may include a series of values obtained during a certain calibration time interval. When the calibration model determination results to be compared are data that is measured at the same time (during a calibration time interval), the correlation between the calibration model determination results may be accurately calculated.

The sensor modules 110a, 110b, 110c, and 110d may be designed to have different detection characteristics. For example, the sensor modules 110a, 110b, 110c, and 110d may correspond to different concentration ranges of the same gas or may be designed to detect different gas materials. Also, the valid measurable time interval may be set differently for each of the sensor modules 110a, 110b, 110c, and 110d.

The processor 120 may generate sensor characteristic description data for describing the sensor 110 configured to recognize real-world odors. The sensor characteristic description data may include information regarding default specifications and functions of the sensor 110. The processor 120 may generate calibration characteristic description data including information regarding whether online or on-site self-calibration corresponding to degradation caused by aging can be performed on the sensor 110. Expression data for describing characteristics of the sensor 110 may include sensor characteristic description data and calibration characteristic description data.

The processor 120 may include, in the calibration characteristic description data, coded information for identifying a calibration type and a calibration method. For example, according to an embodiment of the present invention, the processor 120 may include, in the calibration characteristic description data, an identification code indicating that a function of calibrating the olfactory recognition model configured to recognize real-world odors on the basis of a result of the comparison between the calibration model determination results using the calibration model is provided by the sensor 110.

Each of the sensor characteristic description data and the calibration characteristic description data is described in a standardized manner that has an XML format or the like and that is compatible between heterogeneous platforms.

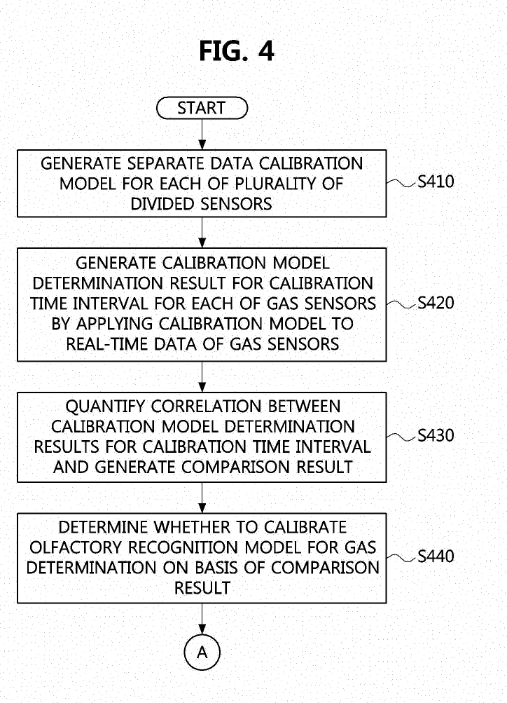

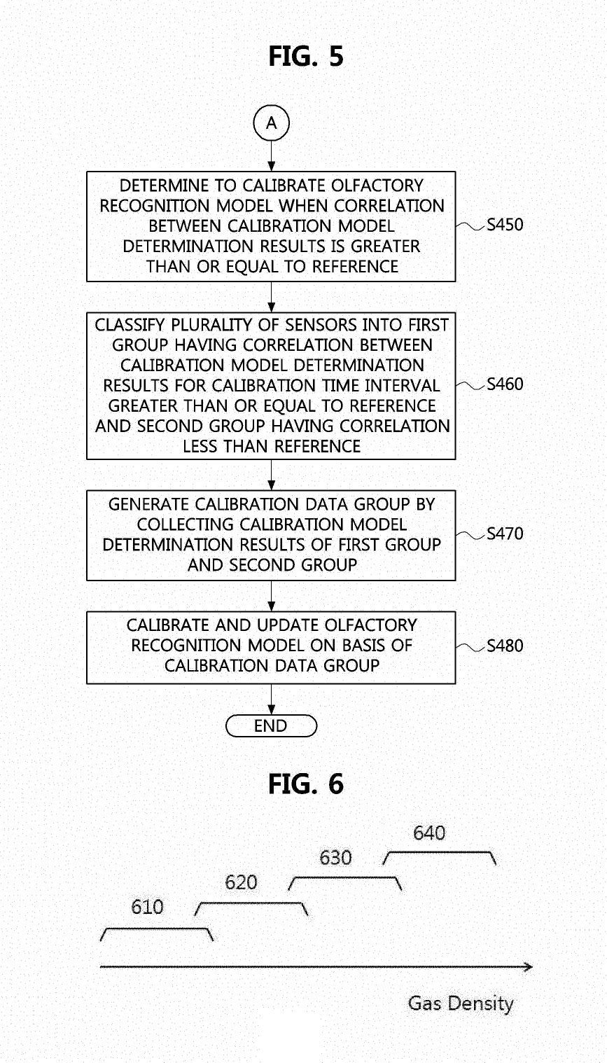

FIGS. 4 and 5 are flowcharts showing an olfactory information generation method according to an embodiment of the present invention. The olfactory information generation method shown in FIGS. 4 and 5 may be performed by the processor 120 of FIG. 1. In particular, the method is described in the form of computer program instructions, and thus the computer program instructions may be loaded to the processor 120 of FIG. 1 and executed by the processor 120 to perform the method.

Referring to FIG. 4, the olfactory information generation method, which generates olfactory information that may be shared between the real world and at least one virtual world, may include generating a separate data calibration model for each of a plurality of divided sensors (S410).

A calibration model determination result for a calibration time interval is generated for each of the gas sensors by applying a calibration model to real-time data of gas sensors (S420).

A comparison result is generated by quantifying a correlation between the calibration model determination results for the calibration time interval (S430).

Whether to calibrate an olfactory recognition model for gas determination is determined on the basis of the comparison result (S440).

Referring to FIG. 5, the olfactory information generation method includes determining to calibrate the olfactory recognition model when the correlation between the calibration model determination results is greater than or equal to a reference (S450).

The plurality of sensors are classified into a first group having the correlation between the calibration model determination results for the calibration time interval greater than or equal to the reference and a second group having the correlation less than the reference (S460).

A calibration data group is generated by collecting the calibration model determination results of the first group and the second group (S470).

The olfactory recognition model is calibrated and updated on the basis of the calibration data group (S480).

FIG. 6 is a diagram showing gas concentration areas corresponding to a plurality of gas sensors according to an embodiment of the present invention.

A first concentration interval 610 is an interval of concentration detectable by the first sensor module 110a, and a second concentration interval 620 is an interval of concentration detectable by the second sensor module 110b. A third concentration interval 630 is an interval of concentration detectable by the third sensor module 110c, and a fourth concentration interval 640 is an interval of concentration detectable by the fourth sensor module 110d. In this case, an overlap interval between the first concentration interval 610 and the second concentration interval 620 may affect a correlation between calibration models of the first sensor module 110a and the second sensor module 110b. That is, when the overlap interval is greater than or equal to a certain level, it is possible to easily calculate a correlation between both sensor modules. FIG. 6 shows an example in which different gas sensor modules correspond to different concentration intervals, but the present invention is not limited thereto. The gas sensor modules may be configured to detect different gas materials. In this case, there will be almost no overlapping area. The correlation is a consistent measurement tendency between the different gas sensor modules. That is, a sensor showing a heterogeneous tendency may be filtered out by performing pattern analysis on whether data measured during the same time interval shows the same tendency, and the sensor may be recognized as a sensor that needs to be calibrated.

However, although the sensor that needs to be calibrated is recognized, the calibration is not always possible. When at least two sensors other than the sensor to be calibrated have a constant tendency and a correlation therebetween is greater than or equal to a reference, data requirements needed for calibration may be regarded as being satisfied.

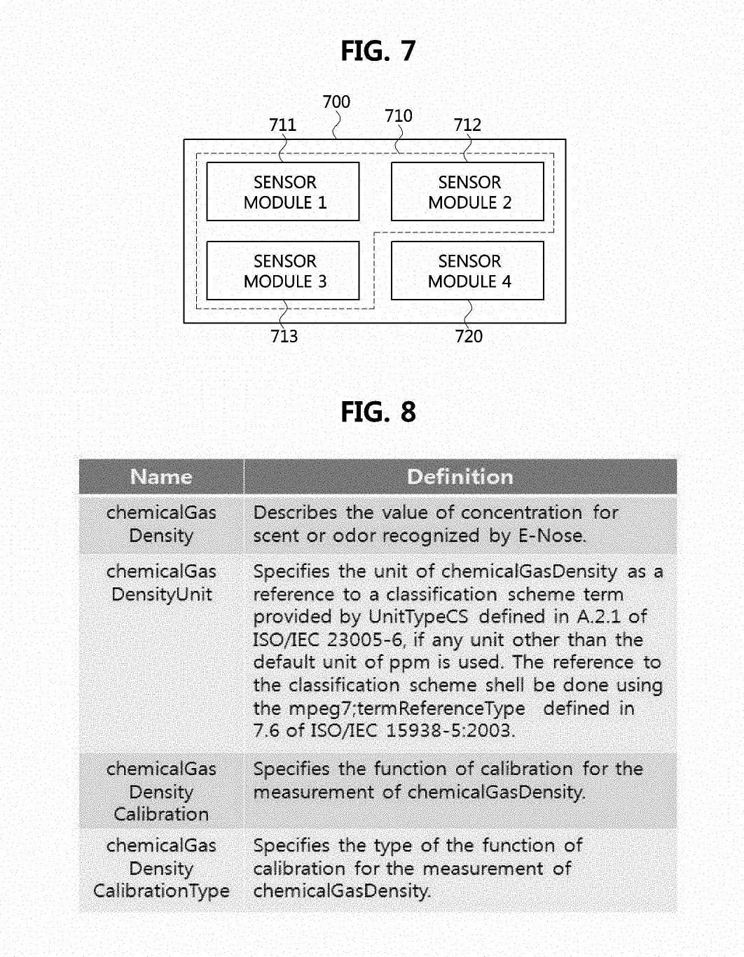

FIG. 7 is a diagram showing a process in which a plurality of gas sensors are formed as a first group and a second group according to a degree of similarity between determination results and a determination result of the second group is calibrated, according to an embodiment of the present invention.

Four sensor modules 711, 712, 713, and 720 in the sensor 700 are classified into a first group 710 having a correlation greater than or equal to a reference as a result of comparing calibration model determination results with each other and a second group. The second group is not shown separately because the second group is composed of only the fourth sensor module 720.

The three sensor modules 711, 712, and 713 are included in the first group 710. Since a correlation therebetween is greater than or equal to the reference, it is possible to generate reference data showing a constant tendency.

Since the only fourth sensor module 720 in the second group has a degree of similarity less than the reference, the fourth sensor module 720 is selected to be calibrated. A calibration data group for calibrating the fourth sensor module 720 may be obtained to calibrate a determination result of the fourth sensor module 720 using a determination result in which the three sensor modules 711, 712, and 713 in the first group 710 have a constant tendency.

FIG. 8 is a diagram in which “Semantics of the EnoseSensorType” of semantics of the Enose Sensor Type are suggested according to an embodiment of the present invention. FIG. 8 is a diagram in which definitions of sub-items of the EnoseSensorType are suggested according to an embodiment of the present invention. EnoseSensorType may define a physical sensor type of an e-nose, but may include all information regarding the e-nose, including detected information. In FIG. 8, items such as chemicalGasDensityCalibration, chemicalGasDensityCalibrationType, etc. are introduced in addition to items such as chemicalGasDensity, chemicalGasDensityUnit, etc. That is, coded information regarding whether a calibration function for a measured gas concentration is held and which type the calibration function has when the calibration function for the measured gas is held may be expressed.

According to an embodiment of the present invention, it is possible to provide interoperability between a virtual world and a real world by recognizing real-world odors within the scope of MPEG-V and delivering the real-world odors to a virtual world.

The present invention is configured to digitalize the type of an odor that is actually detected through olfaction, a time needed for detection, fatigability of a human olfactory organ, etc. and express the digitalized information to correspond to actions of the human olfactory organ. This may contribute to commercialization of research on digitalization of virtual reality, human five senses such as scent display, etc.

According to an embodiment of the present invention, it is possible to generate and deliver detailed information while delivering real-world odors to a virtual world. According to an embodiment of the present invention, it is possible to generate a gas determination pattern recognition model for receiving an input of a measurement value of a gas sensor and estimating a true determination result and also to calibrate the gas determination pattern recognition model and discover an accurate gas determination result.

According to an embodiment of the present invention, processors included in gas sensors may identify a gas sensor that needs to be calibrated by using a pattern recognition model, determine when the gas sensor will be calibrated, and calibrate the gas sensor using a correlation between a plurality of gas sensors, without collecting the gas sensor in an offline state. That is, according to an embodiment of the present invention, it is possible to provide an olfactory information generation apparatus configured to determine the necessity and time of calibration through self-learning of a gas sensor and calibrate a measurement value.

The method according to an embodiment of the present invention may be implemented in the form of a program instruction executable by a variety of computers and recorded on a computer-readable medium. The computer-readable medium may include any one or a combination of a program instruction, a data file, a data structure, etc. The program instruction recorded on the medium may be designed and configured specifically for an embodiment or can be publicly known and available to those skilled in the field of computer software. Examples of the computer-readable medium include a magnetic medium such as a hard disk, a floppy disk, and a magnetic tape, an optical medium such as a compact disc read-only memory (CD-ROM), a digital versatile disc (DVD), etc., a magneto-optical medium such as a floptical disk, and a hardware device specially configured to store and perform program instructions, for example, a read-only memory (ROM), a random access memory (RAM), a flash memory, etc. Examples of the program instructions include not only machine code generated by a compiler or the like but also high-level language codes that may be executed by a computer using an interpreter or the like. The above exemplary hardware device can be configured to operate as one or more software modules in order to perform the operation of an embodiment, and vice versa.

Although the present disclosure has been described with reference to specific embodiments and features, it will be appreciated that various variations and modifications can be made from the disclosure by those skilled in the art. For example, suitable results may be achieved if the described techniques are performed in a different order and/or if components in a described system, architecture, device, or circuit are combined in a different manner and/or replaced or supplemented by other components or their equivalents.

Accordingly, other implementations, embodiments, and equivalents are within the scope of the following claims.

What is claimed is:

1. An olfactory information generation method for generating olfactory information suitable for sharing between a real world and at least one virtual world, the olfactory information generation method comprising:
generating a calibration model determination result of each of a plurality of gas sensors included in a sensor device configured to recognize real-world odors by applying a calibration model allocated to each of the plurality of gas sensors to measurement data of each of the plurality of gas sensors;
comparing the calibration model determination results of the plurality of gas sensors with each other; and
determining whether to calibrate an olfactory recognition model for recognizing the real-world odors, based on a result of the comparison between the calibration model determination results.

2. The olfactory information generation method of claim 1, wherein:
the comparing the calibration model determination results of the plurality of gas sensors with each other comprises calculating a correlation indicating a degree of similarity between the calibration model determination results of the plurality of gas sensors; and
the determining whether to calibrate an olfactory recognition model comprises determining to calibrate the olfactory recognition model when the correlation is greater than or equal to a reference.

3. The olfactory information generation method of claim 2, further comprising determining, among the plurality of gas sensors, a first group including at least two gas sensors having a correlation greater than or equal to the reference and a second group including at least one gas sensor having a correlation less than the reference, wherein
the determining whether to calibrate an olfactory recognition model comprises generating a calibration data group of the olfactory recognition model by using calibration model determination results and measurement data of the gas sensors included in the first group and the second group.

4. The olfactory information generation method of claim 3, wherein the generating a calibration data group of the olfactory recognition model comprises generating the calibration data group including a pattern for calibrating the calibration model determination result of the second group by using a difference between the calibration model determination result of the first group and the calibration model determination result of the second group.

5. The olfactory information generation method of claim 1, further comprising:
generating a calibration data group of the olfactory recognition model by using the calibration model determination results and the measurement data of the plurality of gas sensors when the olfactory recognition model is determined to be calibrated; and
updating the olfactory recognition model by calibrating the olfactory recognition model using the calibration data group.

6. The olfactory information generation method of claim 1, wherein the generating a calibration model determination result of each of the plurality of gas sensors comprises applying the calibration model allocated to each of the plurality of gas sensors to a series of measurement data of the plurality of gas sensors during a first reference time interval to generate the calibration model determination result of each of the plurality of gas sensors including a series of values.

7. The olfactory information generation method of claim 1, wherein the plurality of gas sensors have different detection characteristics, and the detection characteristics includes at least one of a concentration range, a type of a detectable gas material, and a valid measurable time interval.

8. The olfactory information generation method of claim 1, further comprising:
generating sensor characteristic description data for describing the sensor device configured to recognize real-world odors;
generating calibration characteristic description data indicating that a determination result for the real-world odors is capable of being calibrated, based on the measurement data with respect to the sensor device; and
generating expression data for describing characteristics of the sensor device including the sensor characteristic description data and the calibration characteristic description data.

9. The olfactory information generation method of claim 8, wherein the calibration characteristic description data includes a field indicating that the olfactory recognition model for recognizing the real-world odors is capable of being calibrated, based on the result of the comparison between the calibration model determination results with respect to the sensor device.

10. An olfactory information generation apparatus configured to generate olfactory information suitable for sharing between a real world and at least one virtual world, the olfactory information generation apparatus comprising:
a plurality of gas sensors configured to recognize real-world odors and acquire raw data of a result of recognizing the real-world odors; and
a processor configured to generate a calibration model determination result of each of the plurality of gas sensors by applying a calibration model allocated to each of the plurality of gas sensors to measurement data of each of the plurality of gas sensors, compare the calibration model determination results of the plurality of gas sensors with each other, and determine whether to calibrate an olfactory recognition model for recognizing the real-world odors, based on a result of the comparison between the calibration model determination results.

11. The olfactory information generation apparatus of claim 10, wherein the processor calculates a correlation indicating a degree of similarity between the calibration model determination results of the plurality of gas sensors and determines to calibrate the olfactory recognition model when the correlation is greater than or equal to a reference.

12. The olfactory information generation apparatus of claim 10, wherein the processor generates a calibration data group of the olfactory recognition model by using the calibration model determination results and the measurement data of the plurality of gas sensors when the olfactory recognition model is determined to be calibrated, and updates the olfactory recognition model by calibrating the olfactory recognition model using the calibration data group.

13. The olfactory information generation apparatus of claim 10, wherein the processor applies the calibration model allocated to each of the plurality of gas sensors to a series of measurement data of the plurality of gas sensors during a first reference time interval to generate the calibration model determination result of each of the plurality of gas sensors including a series of values.

14. A computer-readable recording medium having a program recorded thereon for executing the method of claim 1.

15. A computer-readable recording medium having a program recorded thereon for executing the method of claim 2.

16. A computer-readable recording medium having a program recorded thereon for executing the method of claim 3.

17. A computer-readable recording medium having a program recorded thereon for executing the method of claim 4.

18. A computer-readable recording medium having a program recorded thereon for executing the method of claim 5.

19. A computer-readable recording medium having a program recorded thereon for executing the method of claim 6.

20. A computer-readable recording medium having a program recorded thereon for executing the method of claim 7.
